SEQUENCE LISTING (3 sequences)

SEQ ID NO: 1 - DNA, 22 bp, Artificial Sequence
  Synthetic primer
aatgatacggcgaccaccgaga

SEQ ID NO: 2 - DNA, 22 bp, Artificial Sequence
  Synthetic primer
caagcagaagacggcatacgag

SEQ ID NO: 3 - DNA, 11 bp, Artificial Sequence
  Synthetic target sequence subject to methylation
tcgcagcgcga

(19) United States
(12) Patent Application Publication
(10) Pub. No.: US 2015/0259743 A1
(43) Pub. Date: Sep. 17, 2015

(54) METHODS OF ASSESSING EPIGENETIC REGULATION OF GENOME FUNCTION VIA DNA METHYLATION STATUS AND SYSTEMS AND KITS THEREFOR

(71) Applicant: Roche NimbleGen, Inc., Madison, WI (US)

(72) Inventors: Daniel Lee Burgess, Black Earth, WI (US); Jason Norton, Sun Prairie, WI (US); Todd Richmond, Mercer Island, WA (US); Jennifer Wendt, Madison, WI (US)

(21) Appl. No.: 14/577,680

(22) Filed: Dec. 19, 2014

Related U.S. Application Data
(60) Provisional application No. 61/922,266, filed on Dec. 31, 2013.

(51) Int. Cl. C12Q 1/68

(52) U.S. Cl. CPC C12Q 1/6883; C12Q 2600/154

(57) ABSTRACT

Systems, kits and methods are disclosed for assessing epigenetic regulation of genome function via deoxyribonucleic acid (DNA) methylation status. The systems, kits and methods rely on a covert then capture concept in which unmethylated cytosine residues in a nucleic acid sequence are first converted to uracil residues and then captured for subsequent analysis. The systems, kits and method use a solution-phase capture probe pool having a mixture of at least three (3) types of capture probes. One type is “wobble” probes, in which some cytosine residues in a nucleic acid sequence are randomly assumed to be unmethylated and thus converted to uracil residues, while other cytosine residues are assumed to be methylated and thus conserved as cytosine residues. Moreover, each type of probe can include a mixture of probes that bind/hybridize to one or the other strand of a nucleic acid sequence of interest, thereby improving sequencing depth and reliability.

### FIELD OF THE INVENTION

The disclosure relates generally to epigenetics, and more particularly to systems, kits and methods of assessing epigenetic regulation of genome function via assessing DNA methylation status.

### BACKGROUND OF THE INVENTION

Epigenetics is the study of the epigenome, which includes the functionally relevant, chemical modifications of DNA and chromatin that occur without altering the fundamental nucleotide sequence. The two main components of the epigenome are DNA methylation and histone modification.

Epigenetic modifications regulate expression of genes in DNA and can influence efficacy of medical treatments among individuals by modulating the expression of genes involved in the metabolism and compartmentalization of therapeutic agents, as well as can alter the expression of the therapeutic agents' targets. Aberrant epigenetic changes are associated with many diseases such as, for example, cancer, cardiovascular disease and neurological disease.

DNA methylation was the first discovered epigenetic mark and remains the most studied. In mammals, it primarily involves enzymatic addition of a methyl (—CH₃) group to the carbon-5 position of cytosine residues of a CpG dinucleotide and represses transcription factor binding thereto. As such, highly methylated regions of DNA tend to be less transcriptionally active.

DNA methylation affects dosage compensation, imprinting, genome stability and development (e.g., stem cell differentiation and embryogenesis) in animals. In addition, it has been linked to transposable element silencing and host-pathogen interactions. DNA methylation likewise is important for genomic integrity in plants.

Current methods for assessing DNA methylation status (i.e. the methylome) focus either on individual loci, using methods such as methylation-specific polymerase chain reaction (PCR) or matrix-assisted laser desorption/ionization time-of-flight mass spectrometry (MALDI-TOF-MS), or on a genome-wide scale using microarrays, reduced representation bisulfate sequencing (RRBS) or whole genome shotgun bisulfite sequencing (WGBS). WGBS is particularly attractive because it measures DNA methylation status with single base pair resolution and allows for assessing percent methylation at each methylatable position in a genome. However, it is still expensive to generate such data for the entire genome of multiple individuals when typically only a small fraction of each genome is of interest.

DNA sequencing-based methods of assessing methylation employ chemical treatments (e.g., bisulfite (BS)) to distinguish methylated cytosine residues from unmethylated cytosine residues. Briefly, BS converts cytosine residues in DNA to uracil residues, which are replaced by thymine residues during subsequent amplification or sequencing reactions. 5-methylcytosine (5-mC) and 5-hydroxymethylcytosine (5-hmC) residues, however, are resistant to conversion and thus conserved as cytosine residues. As such, BS conversion introduces specific changes in DNA that depend on the methylation status of individual cytosine residues, yielding single-nucleotide resolution information about the methylation status of a DNA sequence.

Unfortunately, BS conversion requires a large DNA sample (e.g., >10 μg) because the harsh conditions can degrade about 90% of the sample. In addition, it effectively doubles the size of the genome after amplification because the amplification products of the coding (or sense) and non-coding (or antisense) strands are no longer complementary. Furthermore, partial conversion can occur where only some methylatable cytosine residues actually are methylated, thus complicating traditional probe and assay design and confounding subsequent analysis. The complexities introduced by BS conversion have hindered development of targeted DNA enrichment methods that would facilitate the study of DNA methylation.

For the foregoing reasons, there is a need for additional systems, kits and methods for assessing epigenetic regulation of genome function via DNA methylation status.

### BRIEF SUMMARY OF THE INVENTION

The present invention includes a “convert-then-capture” method of assessing DNA methylation status via targeted enrichment sequencing. Advantageously, the convert-then-capture method permits one to use a small amount of DNA without compromising high molecular complexity while achieving high reproducibility, decreases cost and time required per sample and results in improved sequencing coverage depth. The convert-then-capture method also permits assessment by whole genome sequencing (WGS).

The method may begin by obtaining a DNA sample from a target organism. Once the DNA sample is obtained, the methods may include preparing a DNA library from the sample. Then, the methods may include converting unmethylated cytosine residues in the prepared DNA library to uracil residues with a converting agent such as bisulfite (HSO₃⁻). 5-mC residues, however, are not converted to uracil residues. Alternatively, or in addition, the methods may include converting 5-hmC residues in the prepared DNA library to 5-formylcytosine (5-fC) residues with a converting agent such as potassium perruthenate (KRuO₄). The 5-fC residues are an intermediate that then may be converted to uracil residues with bisulfite. Again, 5-mC residues are not converted to uracil residues.

After converting cytosine and 5-hmC residues and amplifying (e.g., by PCR), the methods include capturing the fragments of interest from the converted DNA library with a solution-based capture probe pool as described herein. Following capture, the methods may include amplifying and purifying captured nucleic acid fragments followed by sequencing. Moreover, the methods may include analyzing the sequence to obtain information regarding DNA methylation status, and may further include comparing the sequence and methylation status of the captured nucleic acid fragments to a sequence and methylation status of a reference genome.

In one embodiment, the invention is a solution-phase capture probe pool for capturing a nucleic acid sequence of interest, the probe pool comprising three types of capture probes: a first type is probes that can hybridize to the sequence of interest containing only uracil residues in place of methylatable cytosine residues; a second type is probes that can hybridize to the sequence of interest containing only cytosine residues in place of methylatable cytosine residues; and a third type is probes that can hybridize to the sequence of interest containing uracil residues in place of some methylatable cytosine residues and cytosine residues in place of other methylatable cytosine residues. In variations of this embodiment, the capture probes are about 50 bp to about 150 bp in length, e.g., about 75 bp in length. The probes may have about 50% G+C. Further, within the probe pool, each of the three types of capture probes is about 33% of the probe pool.

In another embodiment, the invention is a method of assessing DNA methylation status of a nucleic acid sequence of interest, the method comprising the steps of: in-solution capturing of converted and amplified nucleic acid fragments of the nucleic acid sequence of interest with a capture probe pool comprising three types of capture probes where: a first type is probes that can hybridize to the sequence of interest containing only uracil residues in place of methylatable cytosine residues; a second type is probes that can hybridize to the sequence of interest containing only cytosine residues in place of methylatable cytosine residues; and a third type is probes that can hybridize to the sequence of interest containing uracil residues in place of some methylatable cytosine residues and cytosine residues in place of other methylatable cytosine residues; amplifying the captured nucleic fragments to obtain a population of amplified captured nucleic acid fragments; sequencing the amplified captured nucleic acid fragments to obtain nucleotide sequences of the captured nucleic acid fragments; and analyzing the nucleotide sequences of the captured nucleic acid fragments to obtain information regarding DNA methylation status. In variations of this embodiment, the method further comprises the initial step of obtaining a genomic DNA sample and preparing a DNA library from the genomic DNA sample. In this embodiment, the converted nucleic acid fragments are obtained by converting unmethylated cytosine residues and/or 5-hyroxymethylcytosine residues to uracil residues in the DNA library with a converting agent, such as apolipoprotein B editing complex catalytic subunit 1, bisulfite, cytosine deaminase, nitrous acid and potassium perruthenate. In other variations, the method further comprises a step of comparing the nucleotide sequences and methylation status of the captured nucleic acid fragments to a nucleotide sequence and methylation status of a reference genome.

In yet another embodiment, the invention is a system for assessing DNA methylation status, the system comprising: a solution-phase capture probe pool kit having three types of capture probes, a first type is probes that can hybridize to the sequence of interest containing only uracil residues in place of methylatable cytosine residues; a second type is probes that can hybridize to the sequence of interest containing only cytosine residues in place of methylatable cytosine residues; and a third type is probes that can hybridize to the sequence of interest containing uracil residues in place of some methylatable cytosine residues and cytosine residues in place of other methylatable cytosine residues; and at least one additional kit selected from the group consisting of a DNA sampling kit, a DNA library preparation kit, a DNA conversion kit, A DNA amplification kit, a DNA sequencing kit, and bioinformatics design and analysis software. The DNA conversion kit may comprise a converting agent selected from the group consisting of apolipoprotein B editing complex catalytic subunit 1, bisulfite, cytosine deaminase, nitrous acid, and potassium perruthenate.

### BRIEF DESCRIPTION OF THE DRAWINGS

FIG. 1 shows a schematic comparing the “convert-then-capture” workflow to the alternative “capture-then-convert” workflow, indicating the serial localization of three molecular bottlenecking steps (triangles) that lead to increased duplication rates in sequence data for the latter and a need for large amounts of input sample DNA.

FIG. 2 is a diagram showing increased target sequence complexity generated by bisulfite (BS) conversion (exemplary sequence SEQ ID NO: 3), which is problematic for probe design and manufacturing when using the convert-then-capture concept.

FIG. 3 is a diagram showing the advantage of using a “wobble” nucleotide to improve manufacturing efficiency and enable the capture of larger and more complex targets than otherwise would be feasible.

FIG. 4 shows performance of the method on two human cell lines.

FIG. 5 shows an experiment comparing different amounts of input DNA.

FIG. 6 shows data obtained from separate samples from the same source to assess reproducibility.

FIG. 7 shows analysis of an in vitro methylated sample.

### DETAILED DESCRIPTION OF THE INVENTION

### Overview

Exemplary systems, kits and methods are provided for assessing (i.e., capturing, sequencing and analyzing) information about DNA methylation status and are based upon the convert-then-capture concept. This concept is in contrast to current methods that largely are based upon first capturing a nucleic acid sequence of interest and then converting unmethylated cytosine residues in the nucleic acid sequence of interest to uracil residues. While the known methods require only a simple set of probes during capture, they unfortunately require a large DNA sample and only provide information about DNA methylation status with respect to a single strand of DNA. This is particularly problematic when, for example, unmethylated cytosine residues are not completely converted to uracil residues or when a cytosine to thymidine (C to T) polymorphism is present in a nucleic acid sequence. In this instance, one loses a benefit of any such information contained in the other strand and obtains incorrect information about DNA methylation status. Overall, the known methods result in high sample input (e.g., >10 μg) and result in incomplete data coverage over the targeted region of interest, low molecular complexity (i.e., high duplicate read rate), increased sequencing costs and poor reproducibility of results. The work described herein therefore is the first to show that the drawbacks noted above can be solved by the convert-then-capture concept. The inventive concept solves the drawbacks via a solution-phase capture probe pool having a mixture of at least three types of capture probes. A probe targeting methylated DNA, unmethylated DNA and a “wobble” probe that due to random incorporation of C or T recognizes both. Moreover, each type of probe can include a mixture of probes that bind/hybridize in solution to one or the other strand of a nucleic acid sequence of interest, thereby improving sequencing depth and reliability. In view of the unique solution-phase capture probe pool, the method of the invention requires a low sample input (e.g., about 1 μg or less), providing high molecular complexity (i.e., low duplicate read rate), high sample throughput and high reproducibility useful for assessing the status of DNA methylation. The systems, kits and methods are useful in a variety of applications, for example diagnostics and research. With respect to diagnostic applications, one of skill in the art can determine an appropriate medical treatment for a subject by assessing whether there are epigenetic changes via aberrant DNA methylation modulating expression of genes involved in the metabolism and compartmentalization of therapeutic or even modulating the expression of the therapeutic agents' targets. In a similar fashion, one of skill in the art can monitor the effect of therapies on DNA methylation patterns to determine treatment efficacy, predict side effects, or detect the emergence of drug resistance. Likewise, one of skill in the art can assess whether a subject has a disease or disorder linked to epigenetic changes via aberrant DNA methylation such as, for example, a cancer, cardiovascular disease and neurological disease. Alternatively, one of skill in the art can identify methylation patterns associated with, or predictive of, normal phenotypic traits in humans or other organisms, including for example agriculturally important animals and plants. Moreover, one of skill in the art can detect changes in methylation patterns in an organism caused by environmental agents, for example toxins that cause deleterious effects by changing gene expression patterns.

With respect to research applications, one of skill in the art can determine the effect of DNA hyper- or hypomethylation on gene expression, chromatin structure and stability as well as epigenetically inherited traits.

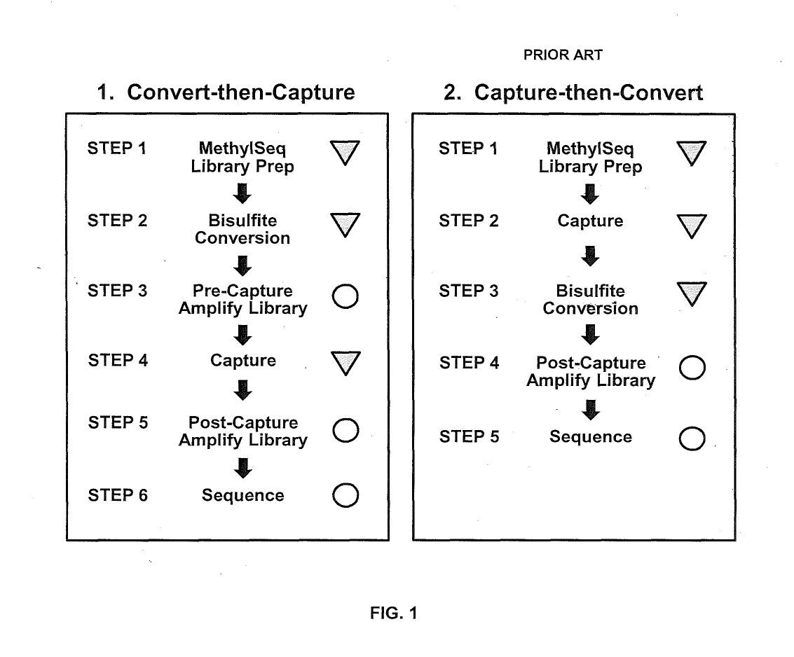

With reference to the drawings, the present invention comprises the convert-then-capture concept. FIG. 1 provides a comparison of the convert-then-capture workflow to the currently practiced capture-then-convert workflow. Workflow steps indicated by filled triangles are steps where a selection process is occurring that decreases sample complexity and therefore information content (a “molecular bottleneck”). For example, in the MethylSeq Library Prep, sample DNA is lost because adapter ligation is only 10%-50% efficient. Likewise, in the BS conversion step, about 90% of DNA is destroyed by the harsh chemical process. Moreover, in the capture step, not all targeted library fragments are captured by probes. The capture-then-convert workflow has the three molecular selection steps in series, which are additive and severely restrict the amount of DNA and information proceeding through the workflow. The convert-then-capture workflow includes the same three steps, but not all in series, so that a PCR amplification step following the first two selection steps (MethylSeq Library Prep, BS conversion) increases the absolute copy number of the library fragments present so that the third selective step (Capture) has less negative impact on sample complexity that would have been caused by sampling from a very small population of molecules. For these reasons, the convert-then-capture approach requires much less sample DNA input at the beginning of the workflow and can allow more information to flow through to the end.

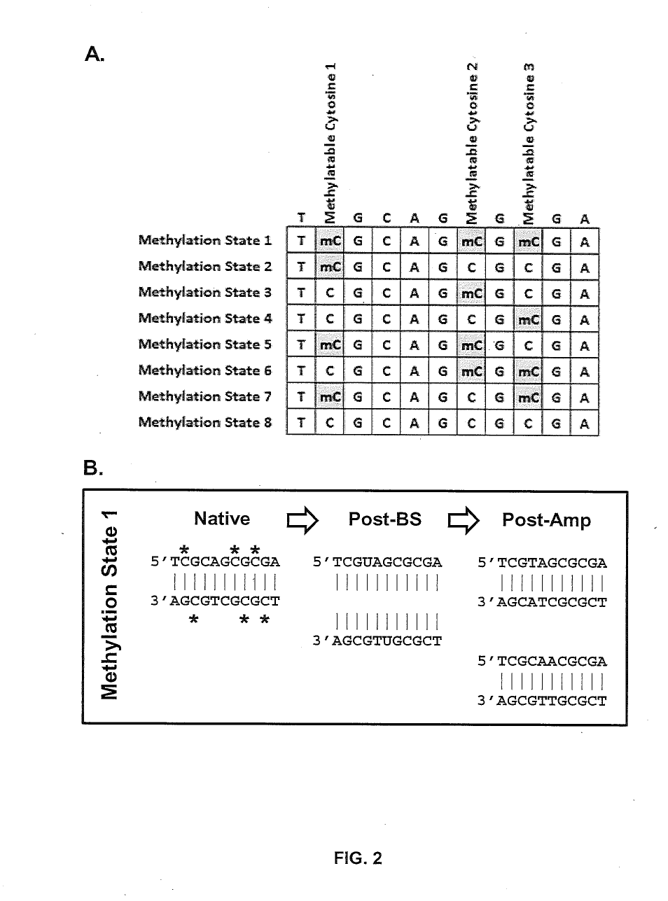

FIG. 2 shows how BS conversion before capture increases target complexity. A hypothetical eleven by capture target is shown having three methylatable cytosines in CpG contexts. Panel A shows that there are eight possible patterns (states) of methylation for this 11 bp sequence. Panel B shows how, after BS conversion and amplification, the daughter strands of the original DNA are no longer complementary and so the number of potential target sequences doubles again, to sixteen. The capture-then-convert concept targets the native DNA where the methylation state of the DNA is irrelevant to capture and so only one (1) probe would be needed to target this locus. In contrast, the convert-then-capture workflow would need sixteen (16) probes.

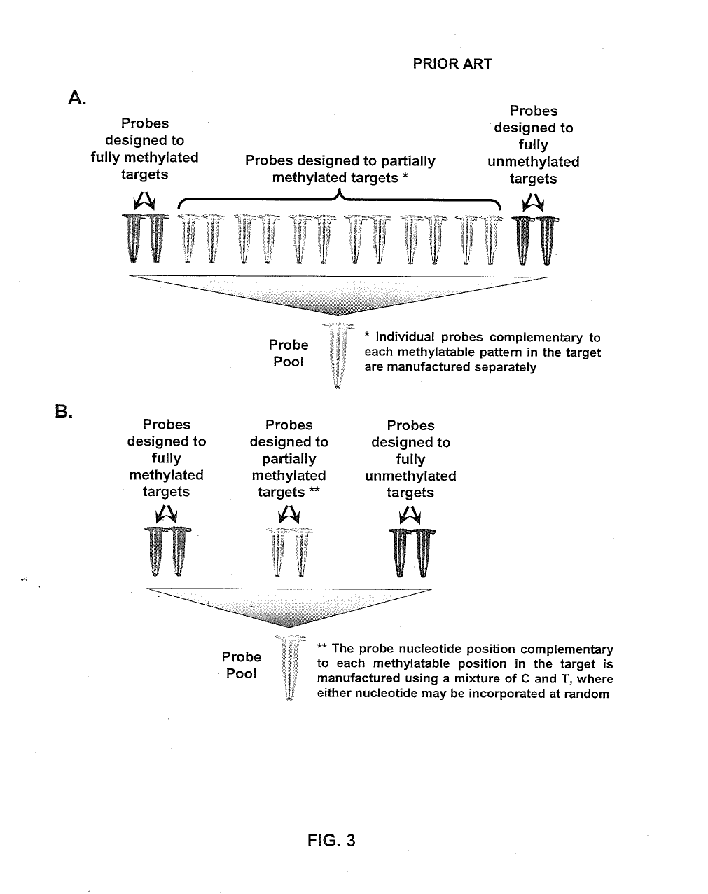

FIG. 3. shows that use of a “wobble” base in oligonucleotide probe manufacture (Panel B) reduces the number of oligonucleotides that must be independently manufactured to match all possible partial methylation patterns of the target sequence. In the existing approach (Panel A), all of the oligonucleotides (probes) needed to match partial methylation patterns are manufactured separately. In the “wobble” approach, the random incorporation of C or T generates the necessary complexity while using far fewer individual oligonucleotide synthesis reactions.

### Systems

Systems of the present invention can include a solution-phase (or in-solution) capture probe pool kit and at least one of the following: a DNA collection or sampling kit; a DNA library preparation/amplification kit; a DNA conversion kit (e.g. for chemical and/or enzymatic treatment to “tag” epigenetic modifications of DNA for subsequent measurement); an amplification/sequencing kit; and bioinformatics design and analysis software.

As used herein, “kit” or “kits” mean any manufacture (e.g., a package or a container) including at least one reagent, such as a nucleic acid probe or probe pool or the like, for specifically amplifying, capturing, tagging/converting or detecting DNA as described herein.

As used herein, “probe” means any molecule that is capable of selectively binding to a specifically intended target biomolecule, for example, a nucleic acid sequence of interest to be bound, captured or hybridized by the probes.

A DNA sampling kit can include components such as syringes, scalpels, cotton swabs, collection, preparation and/or stabilization buffers or stabilizing materials, and sample containers. Kits for collecting or sampling DNA are commercially available from, for example, Bode Technology (Lorton, Va.), DNA Genotek Inc. (Ontario, Canada), Isohelix, Inc. (Kent, United Kingdom) and Norgen Biotek Corp. (Ontario, Canada).

A DNA library preparation and amplification kit can include components such as sequencing adapters, enzymes such as ligases, end-repair enzyme mixes or polymerases, nucleases, PCR primers, buffers, deoxyribonucleotides, ribonucleotides, purification and/or separation columns, beads or matrices, as well as internal controls and quality-control assays for library preparation/amplification. Kits for preparing a DNA library are commercially available from, for example, EMD Millipore Corp. (Billerica, Mass.), Illumina (San Diego, Calif.), Life Technologies (Grand Island, N.Y.), Lucigen (Middleton, Wis.), New England BioLabs Inc. (Ipswich, Mass.), Qiagen (Germantown, Md.), and Roche Molecular Systems (Pleasanton, Calif.). A DNA conversion kit contains reagents for obtaining a converted DNA sample. As used herein, “converted DNA” means a DNA molecule in which one or more unmethylated cytosine residues have been deaminated to become uracil residues. “Converted DNA” means a DNA molecule in which one or more 5-hmC residues have been oxidized to become 5-fmC residues. For example, 5-hmC has been shown to behave like its precursor, 5-mC, during BS conversions. Therefore, BS sequencing data may need to be revisited to verify whether the detected modified base is a 5-mC or 5-hmC residue. These kits can include, but are not limited to, components such as converting agents, lysis buffers, spin columns or other reaction vessels, Proteinase K, other reagents such as DNA protection buffers, and the like. Kits for converting cytosine residues are commercially available from, for example, Life Technologies, New England BioLabs Inc., Qiagen, and Zymo Research (Irvine, Calif.).

The DNA conversion kit can also include components for converting 5-hmC to an intermediate form that is susceptible to conversion with BS to distinguish between 5-mC and 5-hmC residues. These kits can include, but are not limited to, components such as control sequences (e.g., 5-mC and 5-hmC controls), Proteinase K, nucleotides, enzymes such as Msp1 and HpaII, T4 β-glucosyltransferase, DNA polymerase, UDP-glucose, primers, buffers, reaction containers, and the like. Kits for converting 5-hmC residues are commercially available from, for example, Cambridge Epigenetix (Cambridge, United Kingdom), Enzo Life Sciences (Farmingdale, N.Y.), New England BioLabs Inc., and Thermo Scientific (Waltham, Mass.).

A DNA sequencing kit can include components such as enzymes (polymerases, nucleases), primers, dilution, reaction and wash buffers, magnetic beads and nucleotides. Kits for sequencing nucleic acid molecules are commercially available from Affymetrix (Santa Clara, Calif.) Fisher Scientific, Life Technologies, Pacific Biosciences and Qiagen.

The systems can include bioinformatics design and analysis software. See, e.g., US Patent Application Publication Nos. 2006/0014164 and 2010/0161607. The design software can be used to in silico design probes that bind/hybridize with desired specificity to regions of interest in the targeted, converted genome and can include methods for avoiding repetitive regions and utilizing “wobble” bases to address the sequence complexity of the post-amplification converted target sequences. Analysis software can be used, for example, to trim library adapter sequences from the sequence reads output from the experiment, align/map sequence reads to their location in a reference genome, measure methylation rates at individual methylatable sites, analyze data associated with controls included in the system, and identify sequence variants in the sample DNA relative to the reference sequence. Software for analysis of sequence data from BS-converted DNA is commercially available from, for example, Novocraft (Selangor, Malaysia) and CLC bio (Cambridge, Mass.).

As used herein, “methylatable cytosine residue” or “methylatable cytosine residues” mean those residues in the context of CG dinucleotides or in the non-CG contexts of CHG and CHH (where H is an adenine (A), cytosine (C) or thymine (T) residue).

In view of the foregoing, it is contemplated that an exemplary system includes a full complement of a DNA sampling kit, a DNA library preparation/amplification kit, a DNA conversion kit, an amplification/sequencing kit, a solution-phase capture probe pool kit, and bioinformatics design and analysis software.

Positive and negative controls can be included in the kits to validate the activity and correct usage of reagents employed in accordance with the inventive concept. Controls can include samples, such as DNA or RNA preparations from tissues or cell lines, and the like, known to be either positive or negative for the presence of DNA methylation. The design and use of controls is standard and well within the routine capabilities of one of skill in the art.

### Kits

As noted above, kits encompassing the present invention (separately or as a part of the system described above) can include a probe pool for targeted, solution-phase capture of converted DNA having at least three (3) probe types, each of which are directed toward a nucleic acid sequence of interest and target CG, CHG and/or CHH sites in the sequence (where H is an A, C or T residue).

The probes can be synthesized by one of skill in the art, or derived from appropriate biological preparations. Likewise, the probes may be specifically designed to be labeled. Examples of molecules that can be utilized as probes include, but are not limited to, polynucleotides such as RNA or DNA, as well as proteins, antibodies and organic molecules.

Methods of synthesizing polynucleotides for use as probes are well known in the art, such as cloning and digestion of the appropriate sequences, as well as direct chemical synthesis (e.g., ink-jet deposition and electrochemical synthesis). Methods of cloning polynucleotides are described, for example, in Copeland et al. (2001) Nat. Rev. Genet. 2:769-779; Current Protocols in Molecular Biology (Ausubel et al. eds., John Wiley & Sons 1995); Molecular Cloning: A Laboratory Manual, 3^{rd} ed. (Sambrook & Russell eds., Cold Spring Harbor Press 2001); and PCR Cloning Protocols, 2^{nd} ed. (Chen & Janes eds., Humana Press 2002). Methods of direct chemical synthesis of polynucleotides include, but are not limited to, the phosphotriester methods of Reese (1978) Tetrahedron 34:3143-3179 and Narang et al. (1979) Methods Enzymol. 68:90-98; the phosphodiester method of Brown et al. (1979) Methods Enzymol. 68:109-151; the diethylphosphoramidate method of Beaucage et al. (1981) Tetrahedron Lett. 22:1859-1862; and the solid support methods of Fodor et al. (1991) Science 251:767-773; Pease et al. (1994) Proc. Natl. Acad. Sci. USA 91:5022-5026; and Singh-Gasson et al. (1999) Nature Biotechnol. 17:974-978; as well as U.S. Pat. No. 4,485,066. See also, Peattie (1979) Proc. Natl. Acad. Sci. USA 76:1760-1764; as well as EP Patent No. 1721908; Int'l Patent Application Publication Nos. WO 2004/022770 and WO 2005/082923; US Patent Application Publication Nos. 2009/0062521 and 2011/0092685; and U.S. Pat. Nos. 6,521,427; 6,818,395; 7,521,178 and 7,910,726.

Given the complexity and diversity of the probe pool, particularly with respect to the third probe type, a preferred method of synthesizing the probes for the probe pool is by photolithographic techniques.

Two photolithographic techniques are known in the art. In one technique, a photolithographic mask is used to direct light to specific areas of a synthesis surface to effect localized deprotection of photolabile protecting groups (PLPGs). The use of PLPGs, providing the basis for the photolithography-based synthesis of biopolymer (e.g., polynucleotide) microarrays, is well known in the art. Commonly used PLPGs for photolithography-based biopolymer synthesis include, but are not limited to, α-methyl-6-nitropiperonyl-oxycarbonyl (MeNPOC; Pease et al. (1994) Proc. Natl. Acad. Sci. USA 91:5022-5026), 2-(2-nitrophenyl)-propoxycarbonyl (NPPOC; Hasan et al. (1997) Tetrahedron 53:4247-4264), nitroveratryloxycarbonyl (NVOC; Fodor et al. (1991) Science 251:767-773) and 2-nitrobenzyloxycarbonyl (NBOC; Patchornik et al. (1970) 21:6333-6335). See also, U.S. Pat. Nos. 7,598,019; 7,759,513 and 8,445,734.

The “masked” methods therefore include synthesizing polymers utilizing a mount (e.g., a “mask”) that engages a substrate and provides a reactor space between the substrate and the mount. See, e.g., U.S. Pat. Nos. 5,143,854 and 5,445,934.

The other technique is MAS, where light is directed to specific areas of the synthesis surface effecting localized deprotection of the PLPG by digital projection technologies, such as digital micromirror devices (DMDs). See, e.g., Singh-Gasson et al. (1999), supra. A typical DMD employing a solid-state array of miniature aluminum mirrors can pattern about 786,000 to about 4.2 million individual pixels of light. The DMD thus creates “virtual masks” that replace the physical masks used in traditional microarrays.

These virtual masks reflect the desired pattern of ultraviolet (UV) light with individually addressable aluminum mirrors controlled by a computer. The DMD controls the pattern of UV light projected on, for example, a microscope slide in a reaction chamber, which is coupled to a DNA synthesizer. The UV light selectively cleaves a UV-labile protecting group at a precise location where the next nucleotide will be coupled. The patterns are coordinated with the DNA synthesis chemistry in a parallel, combinatorial manner such that up to about 4.2 million unique probe features can be synthesized in a single microarray. See, U.S. Pat. Nos. 5,096,279; 5,535,047; 5,583,688; 5,600,383; 6,375,903; 6,493,867 7,037,659; 7,183,406. 7,785,863; 7,846,660; 8,008,005; 8,026,094; 8,030,056 and 8,415,101; and US Patent Application Publication Nos. 2001/0010843; 2004/0110212 and 2007/0140906. See also, Hornbeck, “Digital Light Processing and MEMs: Reflecting the Digital Display Needs of the Networked Society,” SPIE/EOS European Symposium on Lasers, Optics and Vision for Productivity and Manufacturing I (Besancon, France Jun. 10-14 1996).

MAS therefore eliminates the need for time-consuming and expensive production of exposure masks. It should be understood that the systems, kits and methods disclosed herein may include and/or utilize any of the various probe synthesis techniques described above; however, given the complexity of the third probe pool, MAS on a microarray is the preferred technique.

Once synthesized, the nucleic acid probes are cleaved/removed from the microarray surface and incorporated into the kit. Methods of removing nucleic acid probes from a microarray surface are well-known in the art and can include chemical cleavage, enzymatic cleavage, RNA transcription from DNA oligonucleotide templates and in situ PCR. See, e.g., Saboulard et al. (2005) Biotechniques 39:363-368.

As used herein, “microarray” means a two-dimensional arrangement of features on a surface of a solid or semi-solid support. A single microarray or, in some cases, multiple microarrays (e.g., 3, 4, 5 or more microarrays) can be located on one solid support. The size of the microarrays depends on the number of microarrays on one solid support. The higher the number of microarrays per solid support, the smaller the arrays have to be to fit on the solid support. The microarrays can be designed in any shape, but preferably are squares or rectangles. As used herein, “feature” means a defined area on the surface of a microarray having biomolecules, such as peptides, nucleic acids, carbohydrates, and the like, attached thereto. One feature can contain biomolecules with different properties, such as different sequences or orientations, when compared to other features. The size of a feature is determined by two factors: (1) the number of features on an array, the higher the number of features on an array, the smaller is each single feature; and (2) the number of individually addressable aluminum mirror elements that are used for the irradiation of one feature. The higher the number of mirror elements used for the irradiation of one feature, the bigger is each single feature. The number of features on the microarray is limited by the number of mirror elements (pixels) present in the DMD. The DMD from Texas Instruments, Inc. currently contains 4.2 million mirror elements (pixels). The number of features within one single microarray therefore is currently limited by this number.

As used herein, “solid support” or “semi-solid support” means any solid material having a surface area to which organic molecules can be attached through bond formation or absorbed through electronic or static interactions such as covalent bond or complex formation through a specific functional group. The support can be a combination of materials such as plastic on glass, carbon on glass, and the like, and can be used as the surface for constructing a microarray of the three probe types. The functional surface can be simple organic molecules but can also comprise of co-polymers, dendrimers, molecular brushes, and the like.

As used herein, “plastic” means synthetic materials, such as homo- or hetero-co-polymers of organic building blocks (monomer) with a functionalized surface such that organic molecules can be attached through covalent bond formation or absorbed through electronic or static interactions such as through bond formation through a functional group. Preferably, “plastic” means a polyolefin, which is a polymer derived by polymerization of an olefin (e.g., ethylene propylene diene monomer polymer, polyisobutylene). More preferably, the plastic is a polyolefin with defined optical properties, like Topas® (Topas Advanced Polymers, Inc.; Florence, Ky.) or Zeonor/Ex® (Zeon Chemicals L.P.; Louisville, Ky.).

As used herein, “functional group” means any of numerous combinations of atoms that form parts of chemical molecules, that undergo characteristic reactions themselves, and that influence the reactivity of the remainder of the molecule. Typical functional groups include, but are not limited to, hydroxyl, carboxyl, aldehyde, carbonyl, amino, azide, alkynyl, thiol and nitril. Potentially reactive functional groups include, for example, amines, carboxylic acids, alcohols, double bonds, and the like.

As such, a first type of probe is a probe that can bind one or the other strand of a nucleic acid sequence of interest in which all cytosine residues are unmethylated and thus converted to uracil residues during conversion. The range of probe length can be from about 50 bp to about 150 bp in length and have any nucleotide composition, with a range of about 10% to about 90% G+C.

A second type is probes that can bind one or the other strand of a nucleic acid sequence of interest in which all cytosine residues are methylated and thus not converted to uracil residues. The range of probe length can be from about 50 bp to about 150 bp in length and have any nucleotide composition, with a range of about 10% to about 90% G+C.

A third type is probes that can bind one or the other strand of a nucleic acid sequence of interest in which some cytosine residues are unmethylated, and thus converted to uracil residues, and others are methylated and thus not converted to uracil residues (i.e. “wobble” probes). As used herein, “wobble probe” or “wobble probes” mean those probes in which residues complementary to each methylatable site of CG, CHG and CHH are variably comprised of a cytosine or a thymine residue for each probe molecule. The manufacture of these probes can be accomplished by introducing a mixture of C and T nucleotides (e.g., phosporamidites) when that position of the probe is being synthesized, so that either C or T is incorporated, at random. As such, wobble probes help with capturing DNA fragments that are partially methylated, without the need to separately synthesize all possible probes complementary to all possible partially methylated targets. The range of probe length can be from about 50 bp to about 150 bp in length and have any nucleotide composition, with a range of about 10% to about 90% G+C.

Typically, the nucleic acid sequence of interest targeted by the three probe types can be of any size, e.g., ranging from about 100 base pairs (bp) to about 250 mega base pairs (Mbp). Other components of the capture probe kit include hybridization buffers, blocking reagents (e.g., cot1 DNA, whole genomic DNA from human or other organisms, capture control DNA fragments or clones, adapter-blocking oligonucleotides), PCR primers, enzymes and buffers, DNA purification columns or beads, and streptavidin-coated paramagnetic beads. It is contemplated that other type of probes also can be included in the kit. Examples of other probes include, but are not limited to, control probes. Positive and/or negative controls can be included in the kits to validate the activity and correct usage of reagents employed in accordance with the inventive concept. Controls can include samples, such as DNA or RNA preparations from tissues or cell lines, and the like, either eukaryotic or prokaryotic, known to be either positive or negative for the presence of one or more forms of DNA methylation. The design and use of controls is standard and well within the routine capabilities of one of skill in the art.

### Methods

In view of the foregoing systems and kits, in vitro methods encompassing the inventive concept include assessing DNA methylation status (i.e., capturing, sequencing and analyzing DNA). The methods generally begin by collecting or obtaining a DNA sample from a subject such as an animal or a plant. In some instances, however, the DNA sample may be obtained from a source such as cultured cells or even prokaryotes or viruses. In other instances, the DNA sample may be a synthetic nucleic acid molecule.

As used herein, “sample” means any collection of cells, tissues, organs or bodily fluids in which genomic DNA can be extracted or isolated. Sample likewise can mean a laboratory preparation from which DNA can be obtained. Examples of such samples include specimens of cells, tissues or organs, bodily fluids and smears. The samples can be collected or obtained by a variety of techniques including scraping or swabbing an area, using a needle to aspirate cells or bodily fluids, or removing a tissue sample. When the sample is a bodily fluid, it can include blood, lymph, urine, saliva, aspirates or any other bodily secretion or derivative thereof from which genomic DNA can be isolated. Methods for collecting various body samples or biopsy specimens are well known in the art and need not be described in detail.

Depending upon the sample type, genomic DNA may need to be extracted or isolated from cellular components. Methods of isolating polynucleotides such as DNA are well known in the art. See, e.g., Molecular Cloning: A Laboratory Manual, 3^{rd} ed. (Sambrook et al. eds., Cold Spring Harbor Press 2001); and Current Protocols in Molecular Biology (Ausubel et al. eds., John Wiley & Sons 1995).

After obtaining the isolated DNA sample, the methods may include preparing a DNA library from the DNA sample with methylated (or unmethylated) adapters and a uracil-tolerant polymerase. Methods of preparing DNA libraries for sequencing methylation patterns are well known in the art. See, e.g., Carless (2009) Methods Mol. Biol. 523:217-234; Feng et al. (2011) Methods Mol. Biol. 733:223-238; and Zhang et al. (2009) Methods Mol Biol. 507:177-187. Typically, methods of preparing a DNA library can be divided into the following stages: (1) fragmenting the DNA sample; (2) end-blunting the fragmented DNA sample if necessary; (3) ligating methylated or unmethylated oligonucleotide adapters to nucleic acid sequences of interest; (4) purifying adapter-ligated nucleic acid sequences of interest; and (5) amplifying the purified, adapter-ligated nucleic acid sequences of interest with, for example, a uracil-tolerant polymerase. Methylated adapters preferably are used because they are not affected by the subsequent conversion.

As used herein, “uracil-tolerant polymerase” means an enzyme that can tolerate nucleic acid templates with dUTP (i.e., has reduced amplification bias or has improved read-ahead function) during an amplification (e.g., PCR). Uracil-tolerant polymerases are commercially available from, for example, Cambridge Epigenetix (Cambridge, UK), Enzymatics Inc. (Beverly, Mass.) and Kapa Biosystems (Woburn, Mass.).

The nucleic acid sequence of interest targeted by the three probe types can be from a human genome or any other organism for which partial or complete genomic DNA, or partial or complete transcript sequence, is available or can be inferred from related organisms. Likewise, the nucleic acid sequence of interest targeted by the three probe types can include a coding or regulatory region of one or more genes and, in humans or other vertebrates, generally will include a plurality of CpG sites, especially in or near genes involved in critical pathways.

In the method of the present invention, about 0.5 μg to about 1.0 μg of DNA can be used as a starting material. Control nucleic acids used to monitor the efficacy of, for example, BS conversion or the capture process itself can be added at this point. If not already fragmented, the DNA in the sample can be fragmented to an average size of about 180 bp to about 220 bp using mechanical shearing methods (e.g., sonication). The fragment ends can be repaired to produce blunt-ended, 5′-phosphorylated fragments using mixtures of polymerases and other enzymes (e.g., DNA Polymerase and Klenow Fragment). dAMP can be added to the 3′-ends of the dsDNA library fragments (i.e., “A-tailing”) to facilitate subsequent ligation of methylated library adapters. Methylated dsDNA library adaptors with 3′-dTMP overhangs can then be ligated to A-tailed library fragments.

After preparing the DNA library, the methods may include converting unmethylated cytosine residues to uracil residues in the adapter-ligated nucleic acid sequences of interest via conversion with a converting agent. Methods of converting unmethylated cytosine residues to uracil residues are well known in the art. See, e.g., Frommer et al. (1992) Proc. Natl. Acad. Sci. USA 89:1827-1831; Hayatsu et al. (1970) J. Am. Chem. Soc. 92:724-726; Hayatsu et al. (1970) Biochem. 9:2858-2865; Shapiro et al. (1970) J. Am. Chem. Soc. 92:422-424; and Shiraishi & Hayatsu (2004) DNA Res. 11:409-415. See also, Boyd & Zon (2004) Anal. Biochem. 326:278-280; Callinan & Feinberg (2006) Hum. Mol. Genet. 15:R95-R101; El-Maarri (2003) Adv. Exp. Med. Biol. 544:197-204; Fraga & Esteller (2002) BioTechniques 33:632, 634, 636-649; Grunau et al. (2001) Nucleic Acids Res. 29:E65; Ivanov et al. (2013) Nucleic Acids Res. 41:e72; Laird (2003) Nat. Rev. Cancer 3:253-266; Hayatsu et al. (2004) Acids Symp. Ser. (Oxf) 261-262; Mill et al. (2006) Biotechniques 41:603-607; and Shiraishi & Hayatsu (2004) DNA Res. 11:409-415.

As used herein, “converting agent” means an agent that deaminates cytosine residues to uracil residues. The converting agent thus converts unmethylated cytosine residues to uracil residues but does not convert 5-mC residues. Examples of converting agents include, but are not limited to, Apolipoprotein B Editing Complex Catalytic Subunit 1 (APOBEC1), cytosine deaminase and nitrous acid.

In some instances, such as for distinguishing 5-mC from 5-hmC residues, an additional converting agent can be used. Typically, methods of converting 5-hmC residues can be divided into the following stages: (1) denaturing, (2) converting, and (3) cleaning/purifying the converted nucleic acid sequences. Conversion kits are commercially available for converting 5-hmC to 5-fmC and include, but are not limited to, TrueMethyl™ Kit (Cambridge Epigenetix), BioArray™ 5-hmC Methylation Kit (Enzo Life Sciences), EpiMark® 5-hmC and 5-mC Analysis Kit (New England BioLabs), EpiJET 5-hmC Analysis Kit (Thermo Scientific).

After converting unmethylated cytosine residues (and/or 5-hmC residues) to uracil residues, the converted DNA library can be amplified by ligation-mediated PCR (LM-PCR) using a uracil-tolerant polymerase.

After amplification, the methods can include in-solution capturing of one or more nucleic acid sequences/fragments of interest from the amplified and converted DNA library with a solution-phase capture probe pool kit as described herein. Typically, methods of capturing converted nucleic acid sequences can be divided into the following stages: (1) denaturing, (2) capturing, and (3) purifying/separating. Methods of in-solution capturing are well known in the art and described in, for example, US Patent Application Publication Nos. 2009/0105081 and 2009/0246788.

Converted and captured nucleic acid sequences/fragments then can be amplified. Methods of amplifying nucleic acid sequences are well known in the art. See, e.g., Saiki et al. (1988) Science 239: 487-491; Current Protocols in Molecular Biology (Ausubel et al. eds., John Wiley & Sons 1995); Molecular Cloning: A Laboratory Manual, 3^{rd} ed. (Sambrook & Russell eds., Cold Spring Harbor Press 2001); and PCR Cloning Protocols, 2^{nd} ed. (Chen & Janes eds., Humana Press 2002).

The amplified, captured nucleic acid sequences/fragments can then be sequenced by any methods know to one of skill in the art to study DNA methylation patterns in regions of interest. After being sequenced, the captured fragments can be analyzed to obtain information regarding DNA methylation status, and may further include comparing the sequence and methylation status of the captured nucleic acid fragments to a sequence and methylation status of a reference genome. As noted above, bioinformatics analysis software is well known in the art.

### Examples

### Example 1

### Benchmarking Technical Performance of the Convert-then-Capture Concept

About 0.5 μg to about 1.0 μg of DNA can be used as a starting material. Control nucleic acids used to monitor the efficacy of, for example, BS conversion or the capture process itself can be added at this point. The DNA sample can be fragmented to an average size of about 180 bp to about 220 bp using mechanical shearing methods (e.g., sonication). The fragment ends can be repaired to produce blunt-ended, 5′-phosphorylated fragments using mixtures of polymerases and other enzymes (e.g., DNA Polymerase and Klenow Fragment). dAMP can be added to the 3′-ends of the dsDNA library fragments (i.e., “A-tailing”) to facilitate subsequent ligation of methylated library adapters. Methylated dsDNA library adaptors with 3′-dTMP overhangs can then be ligated to A-tailed library fragments in a reaction that contains ligation buffer, A-tailed DNA, DNA ligase (typically 1 unit), and methylated dsDNA library adaptors with 3′-dTMP overhangs (typically 1-5 μM final concentration). The ligation reaction can be incubated at about 20° C. for about 20 minutes. The adapted library fragments may be purified from buffers, salts and unligated adapters using DNA purification columns or beads.

After preparing the DNA library, the methods may include converting unmethylated cytosine residues to uracil residues in the adapter-ligated nucleic acid sequences of interest via conversion with a converting agent, e.g., Apolipoprotein B Editing Complex Catalytic Subunit 1 (APOBEC1), BS, cytosine deaminase and nitrous acid. In some instances, such as for distinguishing 5-mC from 5-hmC residues, an additional converting agent can be used.

After converting unmethylated cytosine residues (and/or 5-hmC residues) to uracil residues, the converted DNA library can be amplified by ligation-mediated PCR (LM-PCR) using a uracil-tolerant polymerase in a reaction that includes: about 20 ul of converted DNA library, about 25 ul of 2× uracil-tolerant polymerase master mix (contains polymerase, dNTPs and buffer), about 3 ul of a mixture of mixture of two LM-PCR primers (5 uM stock concentration; primer sequences:

5′-AATGATACGGCGACCACCGAGA-3′-SEQ ID NO: 1 and
5′-CAAGCAGAAGACGGCATACGAG-3′-SEQ ID NO: 2)

and about 2 ul of water.
Exemplary thermocycling conditions can be as follows:
Step 1: about 2 minutes at about 95° C.;
Step 2: about 30 seconds at about 98° C.;
Step 3: about 30 seconds at about 60° C.;
Step 4: about 4 minutes at about 72° C.;
Step 5: return to step 2 and repeat eleven (11) times;
Step 6: about 10 minutes at about 72° C.; and
Step 7: hold at about 4° C.

After amplification, the methods can include in-solution capturing of one or more nucleic acid sequences/fragments of interest from the amplified and converted DNA library with a solution-phase capture probe pool kit as described herein. Converted and captured nucleic acid sequences/fragments then can be amplified by PCR in two identical reactions (to keep volumes low), where each reaction can include: about 20 ul of captured DNA library, about 25 ul of 2× uracil-tolerant polymerase master mix (contains polymerase, dNTPs and buffer), and about 5 ul of a mixture of mixture of two LM-PCR primers (5 uM stock concentration:

5′-AATGATACGGCGACCACCGAGA-3′-SEQ ID NO: 1 and
5′-CAAGCAGAAGACGGCATACGAG-3′-SEQ ID NO: 2).

Exemplary thermocycling conditions can be as follows:
Step 1: about 45 seconds at about 98° C.;
Step 2: about 15 seconds at about 98° C.;
Step 3: about 30 seconds at about 60° C.;
Step 4: about 30 seconds at about 72° C.;
Step 5: return to step 2 and repeat fifteen (15) times;
Step 6: about 1 minute at about 72° C.; and
Step 7: hold at about 4° C.

The amplified, captured nucleic acid sequences/fragments can then be sequenced by any methods know to one of skill in the art to study DNA methylation patterns in regions of interest. After being sequenced, the captured fragments can be analyzed to obtain information regarding DNA methylation status.

### Example 2

### Applying the Method of the Invention to a Series of Human Cell Lines

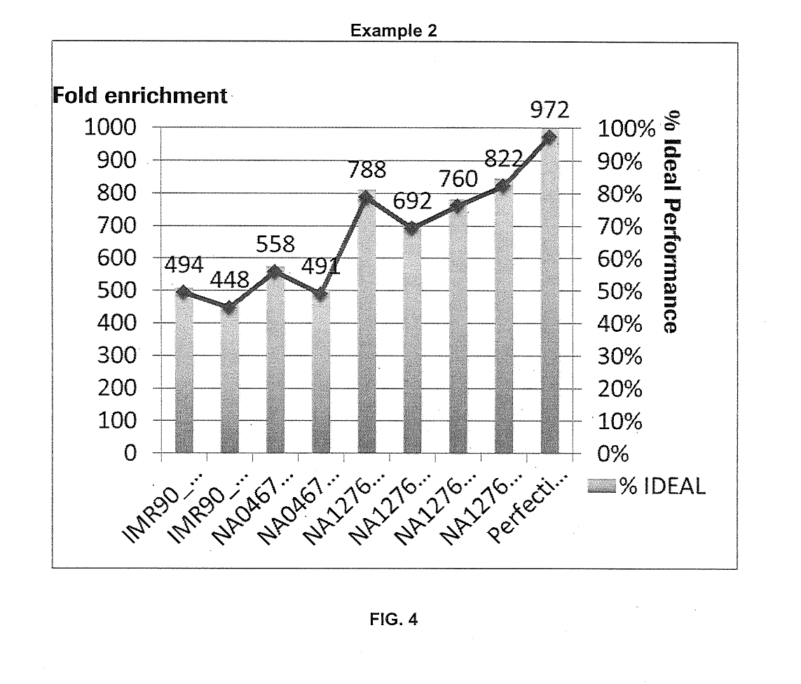

The method as described in Example 1 was applied to DNA isolated from several human cell lines. A 3.2 Mbp capture design was built to regions of interest in human genome hg19. The regions interest included 500 gene promoters across a range of methylation occupancy predicted via roadmap MethylC Seq from cell line IMR90 (normal human lung fibroblast). FIG. 4 shows performance of the capture assay. The assay captured 431 predicted bivalent domains and identified 4 large contiguous imprinted regions in genes CDKN2A, H19-IGF2, XIST and a region on the Y-chromosome.

### Example 3

### Comparing Methylation Patterns in Three Human Cell Lines

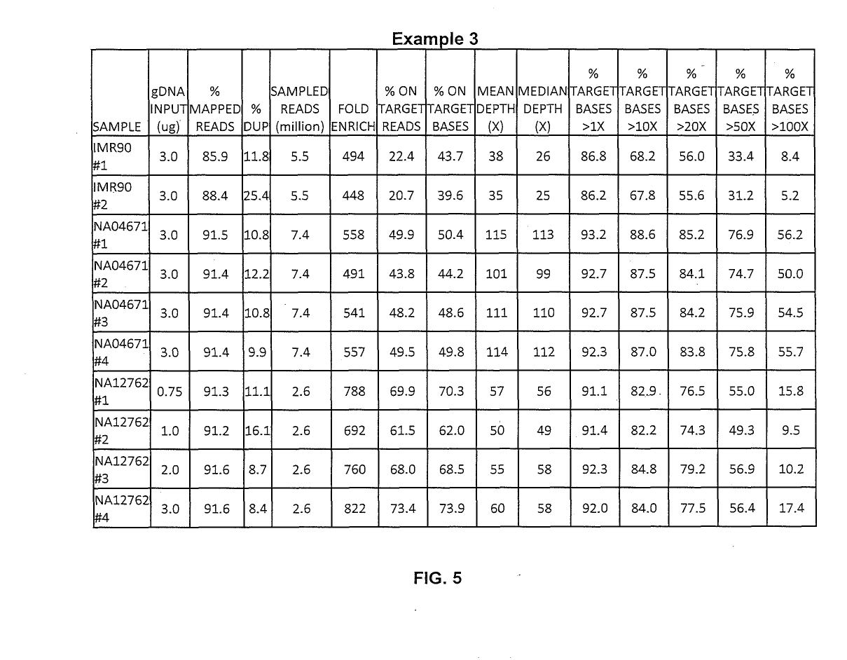

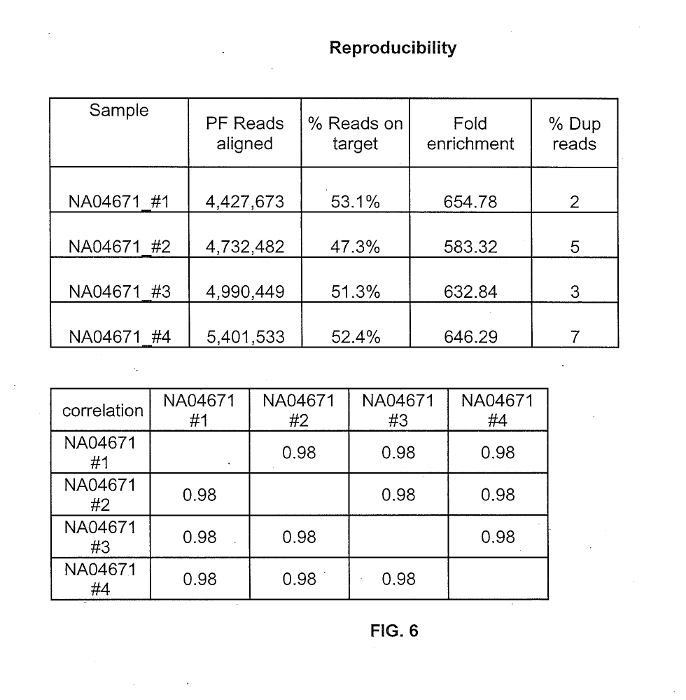

FIG. 5 shows data on identification of methylated sequences in three human cell lines IMR90 (fibroblast), NA04671 (Burkitt's lymphoma) and NA12762 (normal B-lymphocyte). The DNA samples and mixtures thereof where analyzed essentially as described in Example 1. The data shows nearly ideal performance (822-fold enrichment vs. 972-fold maximum possible); low minimum acceptable input (750 ug) of genomic DNA; low duplicate read rate (<10%); >83% coverage of the target space at >10× read depth with only 2.6 M reads. The results indicate 2.5× coverage compared to published data on IMR90 (Lister et al. (2009) Nature 462:315-322). The method further revealed regions hypermethylated and hypomethylated in cancer as compared to the normal cell (data not shown). FIG. 6 shows high reproducibility of data obtained from separate samples from the same source (NA04671).

### Example 4

### Applying the Method to In Vitro Methylated DNA

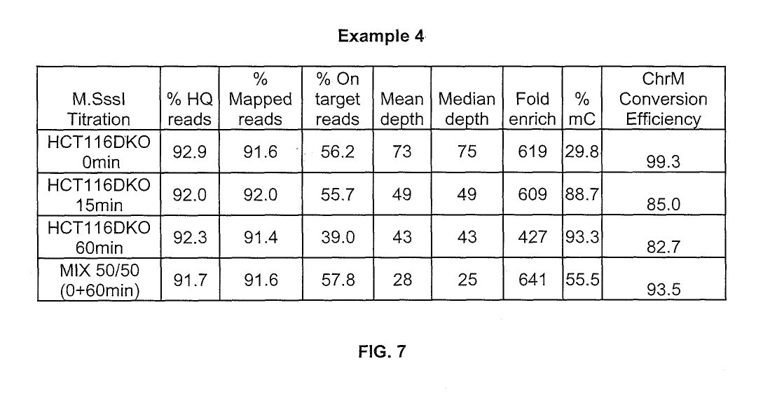

This example utilized a methylation deficient human colorectal carcinoma cell line HCT116 with a double knock out of genes DNMT1 and DNMT3A. DNA isolated from the cell line was incubated with CG methyltransferase for 0, 15 and 60 minutes to obtain various degrees of methylation. The resulting DNA samples and mixtures thereof (a 50/50 mixture of 0+60 minute incubations) were analyzed essentially as described in Example 1. Results on FIG. 7 show that as expected, increased degree of methylation was detected following increased incubation with the CG methyltransferase.

While the invention has been described in detail with reference to specific examples, it will be apparent to one skilled in the art that various modifications can be made within the scope of this invention. Thus the scope of the invention should not be limited by the examples described herein, but by the claims presented below.

We claim:

1. A solution-phase capture probe pool for capturing a nucleic acid sequence of interest, the probe pool comprising three types of capture probes:
wherein a first type is probes that can hybridize to the sequence of interest containing only uracil residues in place of methylatable cytosine residues;
wherein a second type is probes that can hybridize to the sequence of interest containing only cytosine residues in place of methylatable cytosine residues; and
wherein a third type is probes that can hybridize to the sequence of interest containing uracil residues in place of some methylatable cytosine residues and containing cytosine residues in place of other methylatable cytosine residues.

2. The probe pool of claim 1, wherein the capture probes are about 50 bp to about 150 bp in length.

3. The probe pool of claim 1, wherein the capture probes are about 75 bp in length.

4. The probe pool of claim 1, wherein the capture probes have about 50% G+C.

5. The probe pool of claim 1, wherein each of the three types of capture probes is about 33% of the probe pool.

6. A method of assessing DNA methylation status of a nucleic acid sequence of interest, the method comprising the steps of:
(a) in-solution capturing of converted and amplified nucleic acid fragments of the nucleic acid sequence of interest with a capture probe pool comprising three types of capture probes:
a first type is probes that can hybridize to the sequence of interest containing only uracil residues in place of methylatable cytosine residues;
a second type is probes that can hybridize to the sequence of interest containing only cytosine residues in place of methylatable cytosine residues; and
a third type is probes that can hybridize to the sequence of interest containing uracil residues in place of some methylatable cytosine residues and containing cytosine residues in place of other methylatable cytosine residues;
(b) amplifying the captured nucleic fragments to obtain a population of amplified captured nucleic acid fragments;
(c) sequencing the amplified captured nucleic acid fragments to obtain nucleotide sequences of the captured nucleic acid fragments; and
(d) analyzing the nucleotide sequences of the captured nucleic acid fragments to obtain information regarding DNA methylation status.

7. The method of claim 6, wherein prior to step (a), the method comprises the step of obtaining a genomic DNA sample and preparing a DNA library from the genomic DNA sample.

8. The method of claim 6, wherein the converted nucleic acid fragments are obtained by converting unmethylated cytosine residues and/or 5-hyroxymethylcytosine residues to uracil residues in the DNA library with a converting agent.

9. The method of claim 8, wherein the converting agent is selected from the group consisting of apolipoprotein B editing complex catalytic subunit 1, bisulfite, cytosine deaminase, nitrous acid and potassium perruthenate.

10. The method of claim 6, further comprising step (e) comparing the nucleotide sequences and methylation status of the captured nucleic acid fragments to a nucleotide sequence and methylation status of a reference genome.

11. A system for assessing DNA methylation status, the system comprising:
a solution-phase capture probe pool kit having three types of capture probes, a first type is probes that can hybridize to the sequence of interest containing only uracil residues in place of methylatable cytosine residues; a second type is probes that can hybridize to the sequence of interest containing only cytosine residues in place of methylatable cytosine residues; and a third type is probes that can hybridize to the sequence of interest containing uracil residues in place of some methylatable cytosine residues and containing cytosine residues in place of other methylatable cytosine residues; and
at least one additional kit selected from the group consisting of a DNA sampling kit, a DNA library preparation kit, a DNA conversion kit, A DNA amplification kit, a DNA sequencing kit, and bioinformatics design and analysis software.

12. The system of claim 11, wherein the DNA conversion kit comprises a converting agent selected from the group consisting of apolipoprotein B editing complex catalytic subunit 1, bisulfite, cytosine deaminase, nitrous acid, and potassium perruthenate.
